(19) United States
(12) Patent Application Publication
(10) Pub. No.: US 2007/0219630 A1
(43) Pub. Date: Sep. 20, 2007

(54) Devices and Methods for Beating Heart Cardiac Surgeries

(76) Inventor: Xi Chu, Mounds View, MN (US)

Correspondence Address: CRAIG TAYLOR LAW OFFICE, PLLC, 774 RANDY AVE., SHOREVIEW, MN, 55124-2905, US

(21) Appl. No.: 11/577,722

(22) PCT Filed: Nov. 24, 2004
(86) PCT No.: PCT/US04/39418

§ 371 Date: Apr. 28, 2007

(51) Int. Cl. A61F 2/24

(52) U.S. Cl. 623/2.11; 623/2.36

(57) ABSTRACT

The present invention provides devices for beating heart surgery. The device separates the valve and the surrounding area from the rest of the vascular system so the operation procedure can be carried out while the heart is beating during the entire course of the procedure. This is made possible through a temporary valve (170) and two coronary artery conducts (130, 140) incorporated in the balloon-catheter system. The system provides better view and ease of operation and thus, reduces surgery related complication and pains.

### RELATED APPLICATION

The present application claims priority under 35 U.S.C. §119(e) to U.S. filed Oct. 28, 2004, the entire contents of which is incorporated herein by reference.

### FIELD OF THE INVENTION

The present invention relates generally to the field of cardiac surgery such as circulatory valve replacement and repair. More particularly, the present invention relates to the field of beating heart surgeries such as replacement and repair of heart valves through open chest, minimal invasively, or percutaneously. The invention is specifically useful for the replacement of aortic, mitral, tricuspid, and pulmonary valves by prosthetic valves and the repair with or without annuloplasty ring for patients suffering from valve defects such as aortic valve calcification and mitral regurgitation. The operation is supported by a special valved multiple balloon catheter system with or without coronary conduits to separate the operation area from the blood stream and to establish an alternative blood flow for the body, and especially the coronary system without conventional bypass using a heart lung machine. An endoscope, or a fiber optical visualization system is used to supervise the surgery, and to ensure the precisely removal of diseased tissue, repair the defected valve, and attach a new valve at right site. A set of operation tools (kit) such as high speed cutting, aspiration, washing, and valve sewing handles (these tools are interchangeable through the catheter system) and, a pre-attached tissue valve are described thereafter.

### BACKGROUND AND PRIOR ART OF THE INVENTION

Cardiac surgeries represent a large segment of all surgeries performed. Cardiac surgeries correct many heart defects caused by diseases or aging: valve repair and replacement, coronary artery bypass, and heart transplantation. We will use the heart valve replacement and repair as examples to explain the functions of the devices and system subject to this invention. There are four valves in the heart that serve to direct blood flow through the two sides of the heart in a forward direction. On the left side, the mitral and aortic valves direct oxygenated blood coming from the lungs, through the left side of the heart (atrium and ventricle), into the aorta for distribution to the heart itself (through the left and right coronary arteries) and to the rest of the body. On the right side, the tricuspid valve, located between the right atrium and the right ventricle, and the pulmonary valve, located between the right ventricle and the pulmonary artery, direct de-oxygenated blood coming from the body, through the right side of the heart, into the pulmonary artery for distribution to the lungs. The anatomy of the heart and the structure and terminology of heart valves are described and illustrated in detail in numerous references on anatomy and cardiac surgery, including standard texts such as Surgery of the Chest (Sabiston and Spencer, eds., Saunders Publ., Philadelphia) and Cardiac Surgery by Kirklin and Barrett-Boyes, Pathology and Abnormalities of Heart Valves, incorporated herein by reference.

All four heart valves consist of moveable “leaflets” that are designed simply to open and close in response to pressure gradient across the valve. The mitral valve has two leaflets and the triscupid valve has three. The aortic and pulmonary valves are referred to as “semilunar valves” because of the unique appearance of their leaflets, which are most named “cusps” and are shaped somewhat like a half-moon. The components of the mitral valve assembly include the mitral valve annulus; the anterior leaflet; the posterior leaflet; two papillary muscles which are attached at their bases to the interior surface of the left ventricular wall; and multiple chordae tendineae, which couple the mitral valve leaflets to the papillary muscles.

Conventional Open Heart Surgery

Various factors, such as, for example, calcification, may result in the mitrial or aortic valves becoming impaired or functionally inoperative requiring replacement and repair. Where replacement of a heart valve is indicated, in general, the dysfunctional valve is cut out and replaced with either an artificial, synthetic heart valve or a tissue heart valve. The replacement valve is typically sutured in place of the original valve.

It is common to access the heart in a patient's thoracic cavity by making a longitudinal incision in the chest. This procedure, referred to as a median sternotomy includes cutting through the sternum and forcing the two opposing halves of the rib cage to be spread apart allowing access to the thoracic cavity and thus the heart.

Once access to the thoracic cavity has been achieved, surgery on the heart to make valve replacement may be performed. During some procedures, the heart beat is arrested by infusion of a cardioplegic fluid, such as potassium chloride (KCl), to paralyze the myocardium while blood flow circulation is maintained through known heart bypass techniques using a heart-lung machine. Alternatively, the heart is allowed to beat to maintain circulation, while a localized area of the heart, on which surgery is to be performed, is locally immobilized.

The heart is incised and the defective valve is cut away leaving a surrounding area of locally tougher tissue. Known heart valve replacement techniques typically include individually passing individual sutures through the tough tissue to form an array of sutures. Free ends of the sutures are extended out of the thoracic cavity and laid, spaced apart, on the patient's body. The free ends of the sutures are then individually threaded through a sewing ring around the circumference of the replacement valve or a supporting cuff. Once all sutures have been run through the valve, all the sutures are pulled up taut and the valve is slid or “parachuted” down into place adjacent the tough tissue. Thereafter, the replacement valve is secured in place using the sutures.

While the above described procedures are sufficient to successfully install sutures within heart valve tissue, and position an artificial heart valve within the heart and subsequently suture the valve to the tissue, they are particularly time consuming and high cost. In addition, the recovery time is very long and the patients suffer enormous pain and respiring system damage, and even brain damage associated with conventional techniques. Therefore, a need exists for apparatus and procedures of quickly and efficiently suturing artificial heart valves within the heart.

Minimally Invasive Heart Valve Replacement and Repair

Cardiac valve prostheses that need no surgical intervention are known as there are used for implantation by means of a technique of catheterization. Examples of such valve prostheses are described in U.S. Pat. Nos. 3,671,979 and 4,056,854.

U.S. Pat. No. 3,671,979 to Moulopoulos, issued on Jun. 27, 1972, describes a endovascularly inserted conical shaped umbrella-like valve positioned and held in place by an elongated mounting catheter at a supra-annular site to the aortic valve in a nearby arterial vessel. The conical end points toward the malfunctioning aortic valve and the umbrella's distal ends open up against the aorta wall with reverse blood flow, thereby preventing regurgitation.

U.S. Pat. No. 4,056,854 to Boretos, issued on Nov. 8, 1977, describes an endovascularly inserted, catheter mounted, supra-annular valve in which the circular frame abuts the wall of the artery and attached flaps of flexible membrane extend distally in the vasculature. The flaps lie against the artery wall during forward flow, and close inward towards the central catheter to prevent regurgitation during reverse blood flow. The Boretos valve was designed to be positioned against the artery wall during forward flow, as compared to the mid-center position of the Moulopoulos valve, to reduce the stagnation of blood flow and consequent thrombus and embolic formation expected from a valve at mid-center position.

However, both of these valve prostheses are connected to means which lead to the patient either for a subsequent activation of the valve or for a subsequent reposition or removal of the valve prosthesis. With these valve prostheses it is impossible to make an implantation which makes it possible for the patient to resume a substantially normal life in the same way as it is possible in connection with a surgical implantation of a cardiac valve.

U.S. Pat. No. 3,755,823 discloses an elastic stent for a cardiac valve prosthesis. However, this valve prosthesis is not designed for implantation in the body by catheterization. Even though this patent contains no detailed explanation, the description indicates that this valve prosthesis is designed for implantation and sewing on by a surgical intervention.

U.S. Pat. No. 5,545,214 teaches a valve replacement system together with methods of preparation and use for endovascular replacement of a heart valve in a host. The valve replacement system includes up to five components: (1) a prosthetic valve device, (2) a valve introducer device, (3) an intraluminal procedure device, (4) a procedure device capsule, and (5) a tissue cutter. The system provides for endovascular removal of a malfunctioning valve and subsequent replacement with a permanent prosthetic heart valve.

U.S. Pat. No. 5,972,030 to Garrison et al. describes a less-invasive devices and methods for treatment of cardiac valves whereby the need for a gross thoracotomy or median sternotomy is eliminated. In one aspect of the invention, a delivery system for a cardiac valve prosthesis such as an annuloplasty ring or prosthetic valve includes an elongated handle configured to extend into the heart through an intercostal space from outside of the chest cavity, and a prosthesis holder attached to the handle for releasably holding a prosthesis. The prosthesis holder is attached to the handle in such a way that the holder, prosthesis and handle have a profile with a height smaller than the width of an intercostal space when the adjacent ribs are unretracted, preferably less than about 30 mm. In a further aspect, the invention provides a method for repairing or replacing a heart valve which includes the steps of introducing a prosthesis through an intercostal space and through a penetration in a wall of the heart, and securing the prosthesis to an interior wall of the heart, wherein each step is carried out without cutting, removing, or significantly retracting the ribs or sternum.

U.S. Pat. No. 6,010,531 to Donlon et al. describes a less-invasive devices and methods for cardiac valve surgery. Systems and methods are disclosed for performing less-invasive surgical procedures within the heart. A method for less-invasive repair or replacement of a cardiac valve comprises placing an instrument through an intercostal access port and through a penetration in a wall of a vessel in communication with the heart, advancing the instrument into the heart, and using the instrument to perform a surgical intervention on a cardiac valve in the heart under visualization through an intercostal access port. The surgeon's hands are kept outside of the chest during each step. The surgical intervention may comprise replacing the cardiac valve with a prosthetic valve, wherein the native valve is removed using a tissue removal instrument, the native valve annulus is sized with a specialized sizing device, a prosthetic valve is introduced through an intercostal access port and through the penetration in the vessel, and the prosthetic valve is secured at the native valve position, all using instruments positioned through intercostal access ports without placing the hands inside the chest. Systems and devices for performing these procedures are also disclosed.

Beating Heart Cardiac Valve Repair and Replacement

U.S. Pat. Nos. 5,855,614; 5,829,447; 5,823,956; 5,797,960 to Stevens et al. provides devices and methods that facilitate thoracoscopic access into the interior of the heart while the heart is beating. Atrial septal defect (ASD), ventricular septal defect (VSD), and patent ductus arteriosis (PDA). ASD's, VSD's and PDA can frequently be surgically repaired with significant success. Smaller defects may be reparable by simply suturing the defect closed, while larger defects may require a patch of polyester, expanded polytetrafluoroethylene, or a portion of the patient's own pericardium to be sutured into the heart to cover and occlude the defect.

In an effort to avoid the necessity of grossly opening the chest and stopping the heart, a number of intravascular devices have been developed for repair of ASD's, VSD's, and PDA. For example, U.S. Pat. No. 3,874,388 to King et al. discloses an intravascular delivery catheter introduced intraluminally from a peripheral vein into the right side of the heart which can be used to position an artificial umbrella-like patch across a septal defect and to anchor the patch to the cardiac septum. Other intravascular delivery devices and artificial patches for the repair of septal defects can be seen in U.S. Pat. Nos. 5,334,217; 5,284,488; 4,917,089; and 4,007,743.

U.S. Pat. Nos. 5,840,081, and 6,168,614 to Anderson et al. describes a valve prosthesis, which permits implantation without surgical intervention in the body and by using a catheter technique known per se and which makes it possible for the patient to resume a substantially normal life.

This is achieved according to the invention with a valve prosthesis of the type mentioned in the introductory part, which is characterized in that the stent is made from a radially collapsible and re-expandable cylindrical support means for folding and expanding together with the collapsible valve for implantation in the body by means of a technique of catheterization. The collapsible elastic valve is mounted on the stent for instance by gluing, welding or by means of a number of suitable sutures. In connection with the implantation, any prior art technique may be used to supervise an accurate introduction and positioning of the valve prosthesis. Thus, guide wires for the catheter, X-ray supervision, injection of X-ray traceable liquids, ultrasonic measuring, etc. may be used.

U.S. Pat. No. 5,609,598 describes a valve system for treatment of chronic venous insufficiency. The system has inherent limitations in terms of its effectiveness for the procedure described and its applicability, if any, to other valves, especially cardiac valves. U.S. Pat. No. 6,269,819 describes the repair of mitral valve repair U.S. Pat. No. 6,730,118 to Spenser et al. describes a complex catheter based valve replacement

The Disadvantages of Prior Art and the Need for Improvement

All these proposed open heart surgeries such as valve replacement and repair must be performed while the heart is stopped and the bypass circulation is established using a heart-lung machine. In the proposed beating heart surgery, not mention of the separation of the blood to create a better viewable environment, i.e. the operation is conducted in the blood. No consideration of the removal of the calcified valve tissues and the diseased valve, and no description of mounding valve stent to the tissue were presented.

This has two major disadvantages including poor visibility and thus low quality of the operation, and potential stroke and brain damage caused by emboli. In addition, thrombosis and clot will be initiated. Furthermore, for some proposed approaches, no specific consideration for protecting coronary arteries from occlusion by debris during surgery, and no consideration of providing blood to the coronary arteries to ensure the heart functions, which results the immediate death of the patients physiologically.

All these disadvantages have been summarized in a recent review article (Percutaneous Heart

Valve Replacement, Enthusiasm Tempered, Circulation 2004; 110:1876-1878.)

### SUMMARY OF THE INVENTION

To overcome the disadvantages mentioned above, the present invention is directed to apparatuses and methods for the beating heart surgery such as replacement and repair of cardiovascular valves using multi-balloon catheter units designed specifically for each of the four heart valves. Preferably, the multi-balloon unit separates the valve and the surrounding area from the rest of the vascular system so the operation procedure can be carried out while the heart is functioning (Beating Heart) during the entire course of the procedure. This is made possible through the temporary valves and coronary artery conduct (for aortic valve separation) incorporated in the balloon-catheter system. This system provides better view and ease of operation, and therefore reduces surgery related complications. The apparatuses and methods are particularly useful for the aortic, mitral, tricuspid, and pulmonary valves repair with or without annuloplasty ring, or their replacement by prosthetic valves. The main functions of the system include the following aspects.

1. Separating the blood from the operation area so a clear view and a workable space are achieved.

2. Blood flows exact the same paths and the heart is beating during the entire course of the operation without using a heart-lung machine.

3. Beating heart, through a valved multi-balloon unit with or without coronary conduits.

4. Adapt an endoscope or an optical fiberscope so it is a real visualization procedure.

The present invention addresses the needs of all patients with heart valve diseases, including those who heretofore may have been excluded due to being too sick to be candidates for major surgery.

The present invention finds utility not only for the replacement and repair of all heart valves including aortic valves, mitral valves, tricuspid valves, and pulmonary valves, but also other valves such as venous valves of the circulatory system, or other elements in the body systems.

The present invention finds uses not only for the replacement and repair of heart valves but also for providing pure treatment such as gene therapy and calcified tissue treatment, electrophysiological intra-cardiac mapping and ablating etc internal repair such as ASD, VSD, and PDA and many other surgeries.

The invention provides a device for use in beating heart cardiac surgery, which includes a valve separation unit; a visualization system; a surgical system; and a prosthetic valve or valve reinforcement ring element. For example, the aortic valve separation unit comprises a ventricular side aortic valve attachment element; a temporary valve; a connecting tube; a coronary perfusion element; and an ascending aorta attachment element.

The visulization element is selected from a group comprising endoscope, fiberscope, and index matching IR fiber scope.

The surgical system comprises a cutting element, which includes a high speed cutting element and a manual cutter that can be inserted to the desirable locations minimal invasively; a debris removal system having washing and aspiration element; and a suturing and attachment system; a local stabilizer, a material transfer mechanism; and a prosthetic valve. The debris removal system further comprises first fluid introducing element and a fluid aspiration element. The suturing and attachment system can be inserted to the desirable locations minimal invasively, and can be either electrically or manually operated.

The local heart stabilizer will reduce the shaking at the surgical site and the transfer mechanism allows material, tools and prosthetic components to be transferred to the surgical site and the unwanted material waste from the surgical site to the outside of the patient body minimal invasively or percutaneously. The prosthetic valve can be an expandable tissue valve or a U-shaped valve; and the valve reinforcement ring element is an annuplasty ring. The prosthetic valve can be selected from a group comprising an aortic valve, a mitral valve, a tricuspid valve, and a pulmonary valve. The prosthetic valve or valve reinforcement ring element can be pre-mounted on the catheter unit or transferred into the surgical site as an integral device or as sub component minimal invasively or percutaneously.

The invention further provides a method for replacing and repairing cardiovascular valves in a beating heart surgery. The method comprises inserting the device into the heart under a traspeturesous and an optical fiberscope or a thorascope. The device can be inserted into the heart through the chest, the jugular vein, or the femoral artery and vein and advanced percutaneously. The cardiovascular valve is selected from a group comprising an aortic valve, a mitral valve, a tricuspid valve, and a pulmonary valve.

It is an object of the invention to provide devices, systems and kits, and methods for cardiac surgeries with beating heart.

It is an object of the invention to provide a method for the replacement and repair of heart valves to increase the efficiency through the beating heart surgery.

It is a further object of the invention to provide a method for the replacement of aortic, mitral, tricuspid, or pulmonary valve to increase the efficiency of the heart surgery to reduce the patients' pains.

It is also an object of the invention to provide a method for the replacement and repair of heart valves which eliminates the need for cardiopulmonary bypass using heart-lung machine.

It is a further object of the invention to provide for an apparatus for beating heart cardiac surgery such as the replacement or repair of a heart valve during open chest operations. It is a further object of the invention to provide for an apparatus for minimal invasively insertion into the heart to effect the replacement or repair of a heart valve through the right chest via a thorascope, through the jugular vein, through the femoral artery and vein, or through the aorta during close chest operations.

It is a yet further object of the invention to provide a method for the replacement and repair of a valve by percutaneous insertion of valve separation unit, tools, visionlization system, and prosthetic valve or annuplasty ring into the heart to repair and replace a diseased valve

These and other objects of the invention will become apparent to one skilled in the art from the more detailed description given below.

### BRIEF DESCRIPTION OF THE DRAWINGS

FIG. 1a is the schematic representation of a double balloon catheter system employed in the separation and treatment of the aortic valve minimal invasively FIG. 1b is the cross-sectional view of the structure of the double balloon catheter system as shown in FIG. 1a

FIG. 1c is the schematic representation of a double balloon catheter system employed in the separation and treatment for the aortic valve percutaneously. Guide wires are inserted in the coronary artery conduits

FIG. 1d is the schematic representation of a double balloon catheter system employed in the separation and treatment for the aortic valve; including monitor systems such as fiberscope visualization, high speed cutting and suturing elements

FIG. 1e is the schematic representation of a multiple balloon catheter system employed in the separation and treatment for the aortic valve; including a balloon between the first balloon and the coronary artery conduit to expand and attached a prosthetic valve.

FIG. 2 is the schematic representation of a double balloon catheter system employed in the separation and treatment of the mitral valve minimal invasively.

FIG. 3 is the schematic representation of a device system allows the insertion of catheter and surgical instrument into the aorta with beating heart.

FIG. 4 is a schematic representation showing a portion of the human heart including the aortic valve, the left ventricle and an apparatus of the invention in separating the aortic valve and establishing an alternative blood flow.

FIG. 5 is a schematic representation showing portion of the human heart including the aortic, valve, mitral valve, the left ventricle and an apparatus of the invention in separating the mitral valve.

### DETAILED DESCRIPTION OF THE INVENTION

### Part A. Apparatus

The present invention provides devices and methods for use in the beating heart cardiac surgeries. The subject invention provides devices and systems that are capable of separating the aortic valve (and other valves such as pulmonary, mitral, and tricuspid valves) with blood stream and the surrounding area while allowing the blood flow from the left ventricle to the whole body and the coronary arteries so the heart is functioning as a normal one (Beating heart). The employment of the device and methods of the current invention renders the conventional cardiopulmonary bypass using a heart-lung machine unnecessary during the operation. This catheter system is used for the minimal invasive or percutanueous cardiac surgeries such as heart valve repair and replacement. It can be introduced by insertion into the thoracic cavity through the right chest then into the heart via a thorascope. It can also be used percuneously by introducing from the femoral artery and vein or other vessels.

The subject devices, methods, systems and kits find uses in beating heart cardiac surgeries. Devices and methods for their uses in repairing and replacement of the aortic valve, mitral valve and other treatment are provided.

The subject devices include the following system, unit, components, and element:

a valve separation unit;

a fiberscope or enduscope visualization system;

a surgical tool system;

a transfer system;

a heart holding system (stabilizer); and

a prefabricated prosthetic valve, repair ring, or components.

1. Valve Separation Units

Aortic Valve Separation Unit

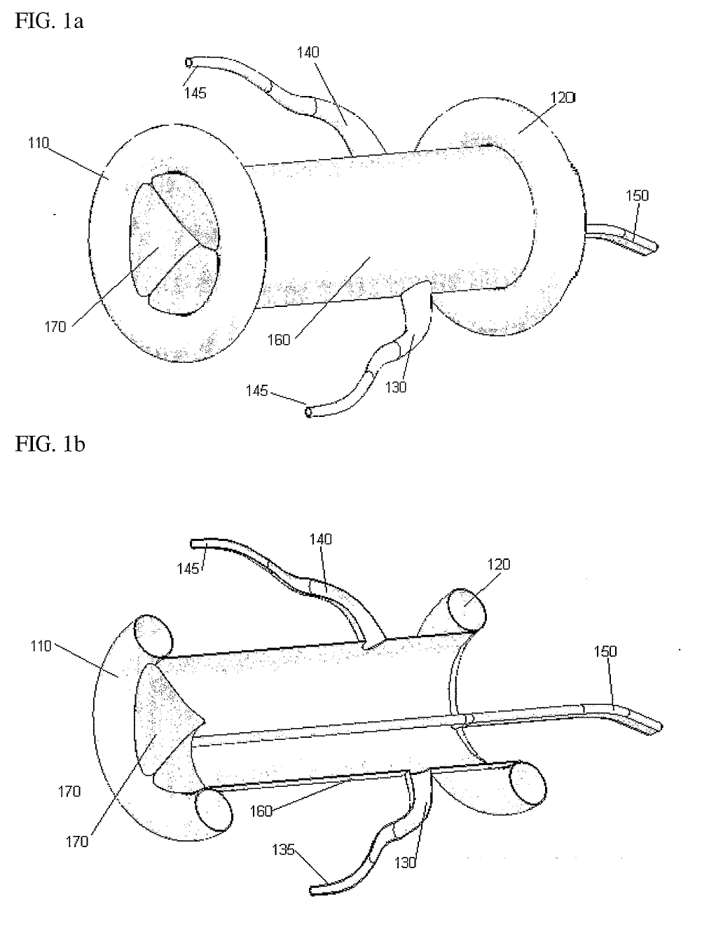

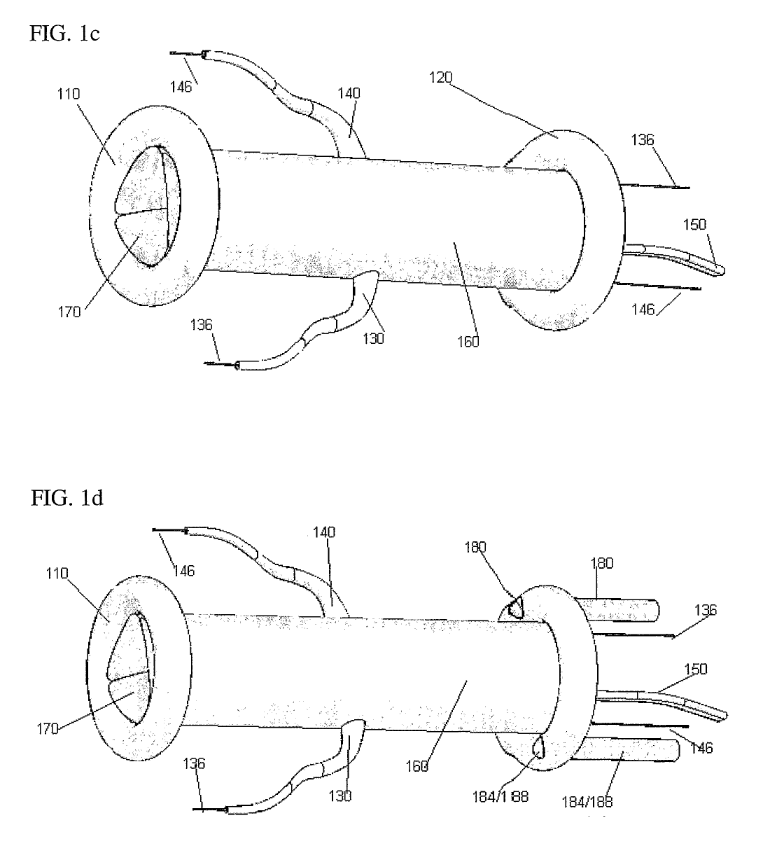

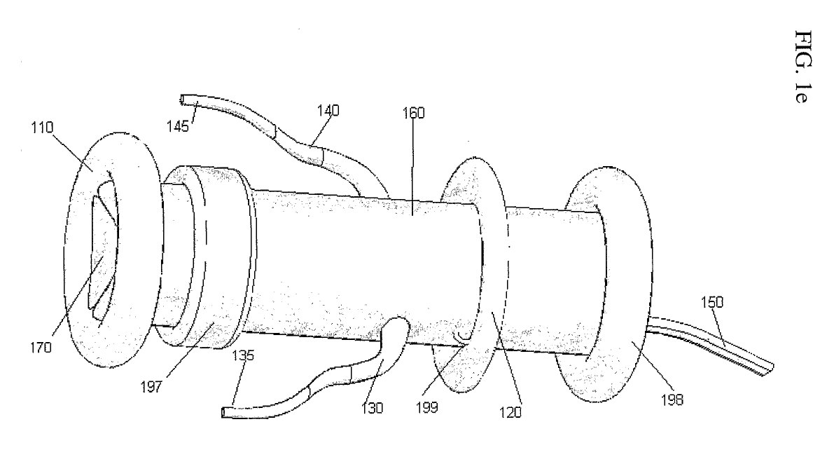

This invention can perhaps be better appreciated by making reference to the drawings. FIG. 1a shows the distal portion of an apparatus of the invention; a double balloon catheter system with two coronary artery conduits as an integral component. Another critical feature is a one way temporary valve similar to aortic valve fabricated at the end of the first balloon catheter system to establish the normal cardiac functions. The system separates the original valve and establishes a workable space around the valve. The two branches of the coronary artery conduits will introduce the blood from the left ventricle to the coronary arteries through the coronary ostia.

The valve separation element of the subject devices is made up of three different sub-elements that work in concert to isolate the target aortic valve to be treated from the remainder of the heart/vasculature of the host. By “separation” is meant that the blood flow between the target aortic valve and the remainder of the vascular system is substantially, if not completely, inhibited. As such, the valve separation system effectively separates the target aortic valve from the remainder of the vasculature. The sub-elements that make up the valve separation element are: (1) a ventricular side attachment balloon 110; (2) temporary valve at the center of the ventricle side balloon 170; and (3) connecting tube 160 between the ventricle side balloon 110 and an ascending aorta attachment balloon 120 and (4) the coronary perfusion conduits 130 140, and (5) an ascending aorta attachment balloon 120.

Each of these elements is now described in greater detail separately.

Ventricular Side Attachment Balloon

The ventricular side attachment balloon 110 serves to occlude blood flow through the original aortic valve by blocking or occluding the upstream side of the valve, i.e., the ventricular side of the valve, while only allow the blood to flow through the temporary valve 170.

This attachment element may be any convenient type of attachment element that can effectively concentrate the ventricular site of the aortic valve. By “effectively concentrate” is meant that fluid, e.g. blood, flow past the gap between the ventricle and the outer wall of the attachment element upon activation is reduced by at least 95%, usually by at least 97% and more usually by at least 99%, where in preferred embodiments, fluid flow is reduced by 100% such that the fluid flow from the ventricle into the center temporary valve is substantially, if not completely.

Representative attachment elements include inflatable balloons, expandable membranes and springs, etc. In many embodiments, the attachment element is an expandable or inflatable balloon. In these embodiments where the attachment element is a balloon, the balloon is generally an expandable balloon that is capable of going from a first, compressed state to a second, expanded state, e.g., by introduction of a fluid or gas into the interior of the balloon, e.g., via an inflation lumen in fluid communication with the interior of the balloon. While the inflatable balloon may be one that is designed to be inflated with a gas or liquid, of particular interest in many embodiments are those that are configured to be inflated with a saline. Balloons suitable for use in vascular devices, e.g., catheter devices, cannula devices, etc., are well known to those of skill in the art and may be readily adapted for use in devices of the present invention. The increase the attachment of the element upon activation, the surface of the balloon might contain some roughness to avoid slip.

Temporary Aortic Valve

At the center of the ventricle side balloon 110, a one-way aortic valve 170 will be constructed to allow the blood flow through the restricted region to the coronary arteries through the two coronary conduits and to the rest of the body. The valve can be molded using polymeric material such as polyurethane or silicone. It has three leaflets 171, 172, and 173 mimic to a nature aortic valve, but services as a temporary role.

Central Connecting Tube and the Coronary Perfusion System

The connecting tube 160 connects the two balloons while providing a central pass way for the blood to flow from the left ventricle through the one-way valve to the coronary arteries through the coronary conduits 130 and 140 defined below and the rest of the body. This element is a thin wall tubing made of blood compatible materials such as polyethylene, silicone, polyureathane, nylon, etc, biocompatible material widely used for blood perfusion. In a preferred embodiment, all the elements are reinforced with high strength fiber. In a further preferred embodiment, the elements can be made in a double-layer form to increase the safety of use during operation.

The next component of the system is the coronary conduits 130 and 140. The conduits introduce blood to the coronary ostia at their opening into the aortic sinuses. More specifically, this perfusion element introduces the flow of blood from the aortic sinuses into the right and left coronary arteries. Some patients have only one coronary artery therefore only one conduit of the device is needed (the other one can be sealed).

This perfusion element may be any convenient tubing element, where representative perfusion elements of interest include, but are not limited to: polyethylene, silicone, polyurethane, nylon, etc, biocompatible material widely used for blood perfusion. The conduits 130 and 140 should have a thin wall and reduced diameter. So they can be folded to minimize their volume before and during delivery into the coronary arteries. After deployment, through the pressure of the blood from the left ventricle, they will be expanded to fit the coronary artery wall.

In many embodiments, the coronary perfusion element is made of two deployable tubes with reduced diameter and foldable and can be delivered into the coronary arteries through the coronary ostia using guide wires similar to the procedure of the coronary stent deployment.

They are dimensioned for insertion into the entrance of the left and right coronary arteries and deployment upon insertion in a manner that introduces substantial amount of blood flow from the left ventricle into the left and right coronary arteries.

Ascending Aorta Attachment Element

The final sub-element of the isolation element is the ascending aorta attachment element 120. This element serves to occlude fluid flow from the ascending aorta in to isolated valve area of its deployment. Its center is connected by the connection element 160 to allow blood flow from the left ventricle to the rest of the body excluding those to the coronary arteries through the perfusion conduits 130 and 140. As such, the ascending aorta attachment element 120 is one that substantially, if not completely, avoids blood flow past its site of deployment from downstream of the aorta back into the isolated aortic valve chamber through the its interface with the internal wall of the aorta.

Therefore, this attachment element is typically deployed at a location before the brachiocephalic trunk, typically at least about 10 mm, usually at least about 10 mm before the brachiocephalic trunk. Representative attachment elements of interest include, but are not limited to: balloons, deployable non-porous membranes, and elastic springs, etc.

In another embodiment, another balloon 125 can be added downstream to 120, but before the brachiocephalic trunk to service as a gate for the transfer system described below in section 3.

Additional Optional Features of the Valve Separation Element

FIG. 1b shows another embodiment of the double balloons catheter that can be inserted through the femoral artery percutaneously during operation. The two coronary artery conduits can be inserted into the coronary arties using the same approaches in coronary stent delivery. After that the two balloons will be inflated to establish the blood flow from the left ventricle to the coronary arteries and the rest of the body. This will separate the aortic valve and the surrounding area providing the space and safety for surgical operation.

FIG. 1c shows additional features of the system. In addition to embodiment described above, there are three access ports in the second balloons for the access of a fiber scope for the visual evaluation (using 188). The other two ports allow the cutting (180) of the diseased tissue, the removal the debris, and the attachment of new valve (184). With two ports, each for specific tool, the surgical time can be reduced.

In another embodiment, port 180 and 184 can be combined to reduce the space and volume in the artery, while the tool can be interchangeable.

FIG. 1d shows additional features of the system. In addition to embodiment described above, there are three access ports in the second balloons for the access of a fiber scope for the visual evaluation (using 188). The other two ports allow the cutting (180) of the diseased tissue, the removal the debris, and the attachment of new valve (184). In another embodiment, another balloon can be added at the section between the two balloons. It will be inflated to attach the new valve to the annulus after the calcified leaflets are removed as will be shown in FIG. 4.

FIG. 1e is the schematic representation of a multiple balloon catheter system employed in the separation and treatment for the aortic valve; including a material transfer ring 199, a third balloon 198 behind the second balloon 120, a fourth balloon 197 between the first balloon 110 and the coronary artery conduit 130,140 to expand and attached a prosthetic valve

Pulmonary Valve Separation Unit

The pulmonary valve separation system is identical to the aortic valve separation system except there is no need for coronary artery conduits. It has two balloons one in the right ventricle, the other in the pulmonary artery before the separation point of the left and right pulmonary arteries. It also has a temporary pulmonary valve at the center of the first balloon. It can be introduced to the location through the chest or the femoral vein

Mitral Valve Separation Unit

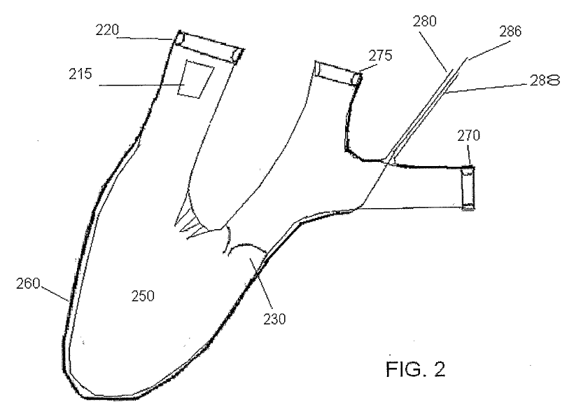

The valve separation unit of the mitral valve is made up of three different sub-elements that work in concert to separate the target mitral valve to be treated from the remainder of the heart vasculature of the host (see FIG. 2). By “separate” is meant that the fluid flow between the target mitral valve and the remainder of the vascular system is substantially, if not completely, inhibited. As such, the valve separation system effectively prevents the target mitral valve from the remainder of the vasculature. The three different sub-elements that make up the valve isolation element are: (1) an aorta side attachment balloon 210; (2) temporary mitral valve 230; and (3) ventricle chamber 250, and (4) atrium side attachment elements 270 and 275 (total of four). Hole 215 is created to allow blood to flow into coronary arteries, assuming that the aortic valve is function well.

Each of these elements is now described in greater detail separately.

Aorta Side Attachment Element

The aorta side attachment balloon 210 serves to attach the device to the inside wall of aorta while allow the central open blood flow through the original aortic valve. If the original aortic valve is functioning well, holes 215 will be created below the attachment balloon and above the aortic valve allow blood flow into coronary arteries.

Representative attachment elements include inflatable balloons, expandable membranes, or elastic metal ring/springs etc. In many embodiments, the attachment element is an expandable or inflatable balloon. In these embodiments where the attachment element is a balloon, the balloon is generally an expandable balloon that is capable of going from a first, compressed state to a second, expanded state, e.g., by introduction of a fluid or gas into the interior of the balloon, e.g., via an inflation lumen in fluid communication with the interior of the balloon. While the inflatable balloon may be one that is designed to be inflated with a gas or liquid, of particular interest in many embodiments are those that are configured to be inflated with a saline. Balloons suitable for use in vascular devices, e.g., catheter devices, cannula devices, etc., are well known to those of skill in the art and may be readily adapted for use in devices of the present invention. The increase the attachment of the element upon activation, the surface of the balloon might contain some roughness to avoid slip. In another embodiment, the color of all elements should be differ from the heart tissue such as red, white to distinguish themselves from various tissue to avoid operational errors.

Temporary Mitral Valve

At the center of the right side branch, a one-way temporary mitral valve 230 will be constructed to allow the blood flow follows the normal path from atrium to ventricle 250 and then to aorta. The valve can be molded using polymeric material such as polyurethane or silicone, but service as a temporary role.

Ventricle Chamber

In many embodiments, the ventricle chamber is made of a large plastic bag. At its inflated or full stage, its surface will has best fit to the internal wall of the left ventricle. To facilitate the expansion after each ventricle contract, an elastic element 260 must be bond to the outer surface of the chamber.

In another embodiment, the elastic element can be the tube use to inflate attachment element 220.

Atrium Attachment Element

The final sub-element of the separation unit is the attachment elements 270 and 275 (total of four, only two are shown). Since the diameters of the left pulmonary vein are small, it is preferred that this ends attached to the interior surface of the atrium through vacuum. These elements serve to prevent blood flow from the pulmonary veins into separated valve area of its deployment. The attachment balloons 220, and the vacuum attachment elements 270 and 275 are connected through tubing 286, 288, and 280 respectively, and they can be inflated or deflated (attach or separate) according to the need similar to the aortic valve separation system.

Additional Optional Features of the Valve Isolation Element

FIG. 2 shows the embodiment of the three balloons catheter that can be inserted through the left atrium minimal invasively during operation. In another embodiment of the catheter that can be inserted through the femoral artery percutaneously during operation. The three tubes connecting to the three balloons will then be collected through the aorta and then femoral artery.

Tricuspid Valve Separation Unit

The tricuspid valve separation unit of the subject devices is similar to the mitral valve separation unit. However, the total number of attachment elements in the right atrium should be three: one for superior vena cava, one for inferior vena cava, and the coronary sinus.

2. Visualization System.

The operation will be supervised by a fiber optical endoscope with wide angles of view. Similar product is commercially available such as SONY fiber optical endoscope system. Another product with the brand name Microfiberscope with diameter from 0.8 to 4 mm and flexible enough to be inserted into the artery system is also commercially available by Imaging Product Group. The scope will be inserted through the catheter and stay during the entire course of the operation to monitor the process and to instruct the cutting of the old valve and the sewing or attaching the new valve/rings and the repair of the valve by rearrange the tissue etc. Other system such as X-ray, ultrasonic, MRI etc can be used as assisting tools.

3. Surgical System

In operating the separated target valve, it is preferred that the pressure in the local environment which includes the isolated target valve (i.e. the area bounded by the ventricular side aortic valve attachment means, the vessel walls of the aortic sinuses and that part of the aortic arch upstream from the ascending aorta attachment means and the ascending aorta attachment means) remains substantially close.

Minimally Invasive Tools and Navigation System

Tools for the minimally invasive operation such as cutting, sewing, and attaching are provided by Intuitive Surgical with commercial system such as da Vinci system. Discussed in the prosthesis valve section in additional to the tool attachment mechanism can be incorporated on the valve so the valve can be attached too the annulus when the old valve is removed through the inflation of the center section of the balloon catheter.

U.S. Pat. No. 5,908,428 Stitching devices for heart valve replacement surgery U.S. Pat. No. 6,197,054 Sutureless cuff for heart valves to perform heart valve replacement surgery is provided. The mechanical heart valve is comprised of a valve body and a plurality of staples extending around the valve body that are coupled to the valve body through at least one intermediate member.

The Debris Removal or Aspiration Element

During the cutting and repair processes, tissue debris will be generated. The system is further characterized in that the debris removal element is attached at its distal end, either directly or through a fluid conveyance linking element, e.g., tube, to a reservoir for waste fluid. In addition, a negative pressure element that provides for suction of fluid from the isolated local environment at the distal end of the fluid removal element into the fluid removal element is also present, where representative negative pressure elements include pumps, vacuums, etc.

In addition to the above fluid introduction and removal elements, in many embodiments the subject devices include a second fluid introduction element for introducing a second fluid into the isolated local environment of the target valve, where the second fluid delivery element is often an element for delivering a dissolution fluid attenuating fluid, as described in greater detail below. When present, the second fluid delivery element may be positioned or configured relative to the above described first fluid delivery and removal elements in a number of different ways. For example, the second fluid delivery element may be a separate tube or analogous structure, where the tube may or may not be present in one or more of the first fluid delivery element or aspiration element, or vice versa, e.g., the different elements may be concentric with each other. Alternatively, the second fluid delivery element may be a lumen present in a multi-lumen structure, where other lumens may be the aspiration and/or first fluid delivery elements.

The second fluid introduction element is further characterized by having a proximal end that is attached, either directly or through a linking fluid conveyance structure, to a source of a second fluid, e.g., a reservoir having a volume of dissolution fluid attenuating fluid present therein, such that the interior of the second fluid introduction means is in fluid communication with a volume of dissolution fluid attenuating fluid. The proximal end of the fluid introduction element typically includes a valve or other flow control element for controlling the amount of the fluid that enters the lumen of the second fluid introduction element from the reservoir of dissolution fluid attenuating fluid.

Transfer Mechanism

FIG. 1e shows that at the second balloon and the central tube, there is a small loop 199, through which, a long suture loop is inserted. The other end of the loop is outside the body for the transfer of needed items, such as a special tool, a leaflet of a tissue valve, a piece of repairing tissue can be transferred to the surgical site if needed.

In another embodiment, material can be transferred through the artery. As described in section 1, at the down stream of the second attachment balloon, there is a third balloon 198. Normally the third balloon is in deflated stage. Once the material is pulled between the second and the third balloons, the third balloon will be inflated. The blood between the two balloons 120 and 198 will be removed, the material and space will be washed. After that, the second balloon will be deflated and the transferred material will be further advanced to the surgical sites. Then, the 2nd balloon 120 will be inflated and the 3^{rd} balloon 198 will be deflated to return to its normal stage.

Heart Local Stabilizer

A local heart stabilizer will be introduced to stabilize the root of aortic valve. This will facilitate the surgery and the suturing of the valve. The device uses at least one rigid beam and at least one negative pressure head to be place at the desirable location.

4. The Prosthetic Valve and Ring

O-shaped and U-shaped valve or ring can be pre-mounted on the balloon. It is preferable that a soft tissue valve will be used and the valve can be folded on the device as assembly. The valve can be a U-shaped i.e. not completely sewed (an aortic valve cut along the interface of two adjacent leaflet) to allow it to be wrapped on the balloon so that the volume is minimized and this provides ease for the insertion and delivery.

The valve may process an attaching mechanism so it will be firmly attach the tissue at right location after deployment. An example is that on the surface of the stent, many fish hook like needles are prewelded. The needles are inserted into the tissue, and the pull-out will be prohibited due to the reverse direction of the hooks.

If the support means are made from a thread structure, this can for instance be loop shaped or helical. This makes it possible to compress the stent and the collapsible valve mounted thereon for placing on the insertion catheter.

5. Additional General Features of the Device

The device may be a device in which all of the elements are statically positioned relative to each other such that no relative movement is possible between any two elements of the device, or two or more of the subject elements may be movable relative to each other in the device. For example, the cleaning and asperiation element may be slidably positioned inside of the fluid removal element; the ventricular side attachment means may be adjustably movable relative to the remainder of the device to provide for an adjustable isolated local environment; etc.

The components of the subject devices, as described above, may be fabricated from any convenient material. The materials must be able to withstand contact with any fluids introduced or removed thereby and should be physiological compatible, at least for the period of time in which they are being used. Suitable materials include biocompatible polymers, e.g. polyimide, polyethylene, and the like. Any glues or fittings that are employed must also be able to meet the same criteria. Any convenient fabrication protocol may be employed, where numerous suitable protocols are known to those of skill in the art.

Also provided by the subject invention are systems for practicing the subject methods, i.e. for cutting a heavily calcified aortic valve with a high speed grinder, e.g., to replace an aortic valve as described above. The subject systems at least include the subject devices as described above, a fluid reservoir for storing washing fluid, and a negative pressure means for providing aspiration or suction during the removing and cleaning of the diseased valve. The systems may further include a number of optional components, e.g. guidewires, pumps for pressurizing the balloon inflation fluid, vacuum for the attachment elements and the like.

In a preferred embodiment, the devices and system should be disposable. Several sizes will be provided to accommodate the wide range of the patient needs. Depending on the specific operation surgery types such minimally invasive or percutaneously, valve repair or valve replacement, the kit contains exchangeable elements.

Also provided by the subject invention are kits for use in treating a patient suffering from congestion heart defects or defects by diseases. The subject kits at least include a device as described above. The kits may further include one or more additional components and accessories for use with the subject devices, including tubing for connecting the various components with fluid reservoirs, syringes, pumping means, etc., connectors, one or more guidewires, dilators, vacuum regulators, spare parts and components etc.

Other elements that may be present in the subject kits include various components of the systems, including manifolds, balloon inflation means, e.g. syringes, pumping means, negative pressure means etc. It is evident from the above discussion and results that improved methods of replacing or repairing the aortic valve are provided. The subject methods and devices provide for significant advantages in the treatment of this. In addition, the subject methods may be less traumatic to the patient that convention valve replacement protocols.

### PART B Operation Methods and Procedures

1. Aortic Valve Replacement and Repair—Minimal Invasive

In practicing the subject methods, a diseased aortic valve is first isolated using the separating system. Then, the isolated valve is repaired or removed with a surgical tool under the surveillance of a fiberscope. In certain embodiments, a prosthetic valve is implanted if necessary. Also provided are systems and kits that include the subject devices and prosthetic valves, and can be employed in practicing the subject methods. Before the present invention is described further, it is to be understood that this invention is not limited to particular embodiments described, as such may, of course, vary.

The subject devices are also characterized in that they are dimensioned to be introduced into the aortic arch through a position upstream before the brachiocephalic trunk and downstream of the root of aortic valve. As such, the size of the devices such as the diameter of the balloon, the distance between the balloons, and the coronary conduits etc) for all patients should be fall in certain groups.

Normally, Cardiac patients will have extensive examination by cardiaologist before the surgery is recommended. Therefore, the size of their internal aorta structure should be known and a proper sized device will be chosen based on these measurements.

As summarized above, the subject devices include an aortic valve separation system, coronary perfusion system, visualization system, a valve surgical element, and a pre-fabricated valve. Depending on the patient's situation, the prosthetic valve might not be necessary in all cases. Each of these systems is now described separately in greater detail.

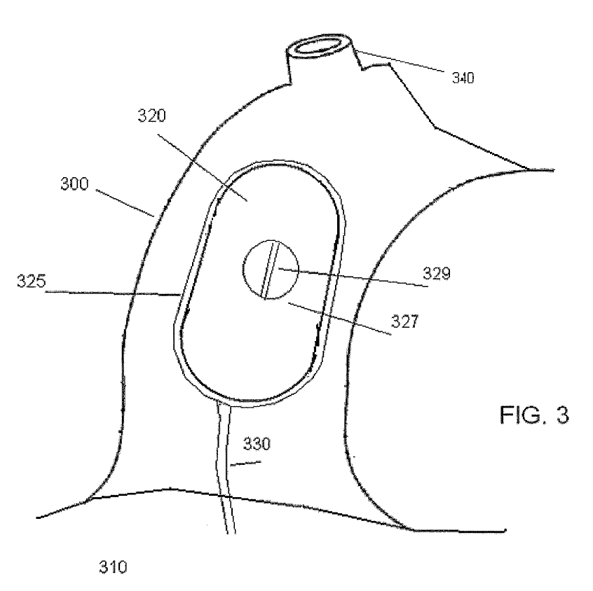

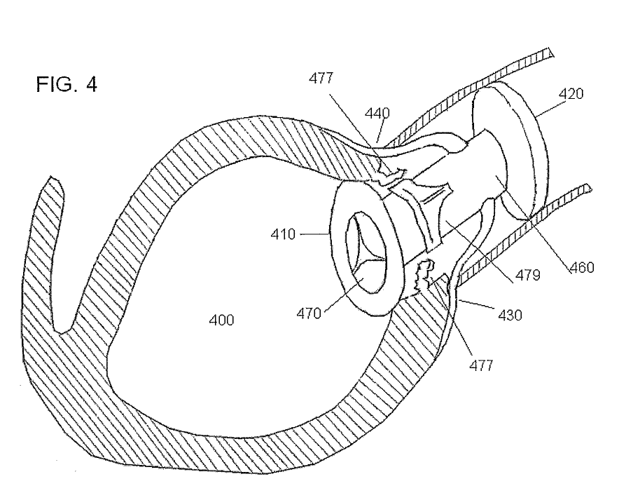

The invention can perhaps be better appreciated by making reference to the drawings. FIG. 3 shows a device that facilitates the insertion of the heart valve separation device described before as shown in FIG. 1. This device is minimal invasively inserted through a small port in the chest to the surface of the aorta. The device is then attached onto the aorta through vacuum at the edge of the cup 325. At the central surface of the device, there is a one way valve 327 to allow instrument to be inserted into the system through the slit 329. During the operation, a knife or scissors will be first inserted through this port to make an incision of the aorta. Then the knife will be retracted. The valve separation device will be inserted through the port and the incision into the aorta. The first balloon will be inserted into the ventricle. The two coronary artery conduits will be first guided into the coronary artery ostia. The two balloons will be inflated and the blood will flow from the left ventricle to pass the temporary valve and then the center connecting tube then through the center of the second balloon during systole. During diastole the blood will flow from the center tube into the coronary arteries the two coronary perfusion conduits. This is functions exactly like a normal heart. Therefore, the aortic valve area is separated from the blood stream. Upon removal of the blood from the area, a clean, viewable and easily accessible and workable space is available for the surgery (as shown in FIG. 4).

In FIG. 4 a portion of the human heart is depicted showing, a left ventricle 400 and the aorta is shown. The original heavily calcified aortic valve 477 is completely separated from the blood stream by the two attachment balloons 410 and 420, and the connecting tune 460. The coronary arteries are perfused by the two conduits 430 and 440. A new prosthetic tissue valve 479 is surrounded on the connecting tube 460 between 410 and the roots of the coronary conduits. Now, regular valve replacement procedure can be performed by removing the original diseased valve, removing the debris, washing the sites and sewing the pre mounted valve 479. The valve could be either a mechanical valve or a tissue one. It should be pre-sleeved on the device between the first balloon and the coronary artery conduits.

Alternatively, to reduce the size of the device the tissue valve can be an un-assembled trileaflet valve, and each leaflet can be transferred to the site and sutured sequentially.

Finally, the incision on the aorta will be closed to leave a small holes with closing suture inserted. The catheter balloon will be deflated and withdrew from the aorta. And the aorta incision is then completely closed. The protection device (FIG. 3) is finally released.

The main advantage of this approach is to reduce the patient's respiration system damage because the heart-lung machine is not used. In addition, surgical related pain will be greatly reduced since the standard median sternotomy is not used.

Method for minimally invasive repairing or replacing a pulmonary valve in a beating heart surgery is in parallel to that for the aortic valve described above except the device is inserted into the pulmonary artery.

2. Aortic Valve Replacement and Repair—Percutaneously

The device and system described in the current invention can also be applied in percutaneous aortic valve replacement and repair. The valve is pre-mounted on a balloon between 110 and coronary conduits 130 and 140 in FIG. 1e and shown in FIG. 4. This is achieved according to the invention with a valve prosthesis of the type, which is characterized in that it can be expanded to attach on the aorta root with attachment mechanism.

The second approach according to the invention with a valve prosthesis of the type, which is characterized in that the stent is made from a radially collapsible and re-expandable cylindrical support means for folding and expanding together with the collapsible valve for implantation in the body by means of a technique of catheterization. The collapsible elastic valve is mounted on the stent for instance by gluing, welding or by means of a number of suitable sutures. If the support means are made from a thread structure, this can for instant be grate shaped, loop shaped or helical.

When the valve prosthesis is introduced and placed correctly, the stent is expanded by self-expansion or by means of the expansion arrangement until the stent is given an outer dimension which is slightly larger than the channel in which it is placed. As the stent is elastic, a contraction of the stent is prevented once it is expanded. The stiffness in the material of the support means contributes to maintain the expanded shape of the stent.

After the expansion is made, the expansion arrangement of the system is contracted and the system can be removed from the channel. The inlet opening can subsequently be closed and the patient will then be able to resume a normal life.

Alternatively, even three individual leaflets can be delivered to the site using the mechanism shown in FIG. 1e and sutured sequentially. The leaflet can be transferred to the site using a long loop made of suture thread. Each leaflet can be tied on one side of the loop. By pulling one the other side of the loop, the leaflet will be advanced to the surgical site to be sutured.

The valve prosthesis according to the invention does not require an actual operation but merely a small intervention to optionally expose the body channel, e.g., a vein, through which the insertion takes place. Thus, patients for whom an operation would be associated with high risk can be offered implantation of, for instance, cardiac valves. After the implantation has taken place, the after-treatment will advantageously be shorter than normal, which means fewer hospital days for the patient. Moreover, it is assumed that it will be possible to implant the valve prosthesis under local anaesthetic.

When the valve prosthesis is used as a cardiac valve prosthesis in the aorta, it is possible to mount it in three positions, such as in the descending part of the aorta of a position between the coronary arteries and the left ventricle of the heart, or in the aorta in a position immediately after the mouth of the coronary arteries.

Even though the cardiac valve preferably is meant to be used for patients suffering from aorta insufficiency and who cannot be offered an open heart surgery, the valve prosthesis can also be used for patents in connection with treatment of aorta stenosis. Some of the patients with aorta stenosis are elderly people who cannot be offered a surgical cardiac operation. The patients are offered balloon dilatation of the aorta stenosis which may result in an aorta insufficiency as a side effect of the treatment.

Furthermore, the stent may be made with a relatively great height and with a cylinder surface which is closed by a suitable material. Thus, a vascular prosthesis known per se is formed wherein the valve is mounted. This may facilitate the implantation of the valve prosthesis, for instance in the arcus aorta. Moreover, the great surface which abuts the inner wall of the channel contributes to ensure the securing of the valve prosthesis in the channel. This embodiment is also suitable as valve prosthesis which is inserted in veins. As veins have relatively thin and weaker walls than arteries, it is desirable that the valve prosthesis has a greater surface to distribute the outward pressure which is necessary to secure the valve prosthesis.

Moreover, the invention relates to a balloon catheter for implanting a valve prosthesis according to the invention and comprising a channel for injection of a fluid for the inflation of the balloon means of the catheter and an insertion cap wherein the balloon means of the catheter and a collapsible valve prosthesis mounted thereon are located during the injection, characterized in that the balloon means are provided with profiled surface which is made to ensure a steady fastening of the valve prosthesis during the withdrawal of the balloon means from the protection cap and the subsequent inflation for the expansion of the stent.

In connection with the implantation, any prior art technique may be used to supervise an accurate introduction and positioning of the valve prosthesis. Use the current invention, the operation can be supervised using the fiberscope. If in the blood stream, other means such as, guide wires for the catheter, index matching IR scope, X-ray supervision, injection of X-ray traceable liquids, ultrasonic measuring, etc. may be used.

With this approach, the aortic valve is pre-inspected after inserting the valved balloon catheter as described in section 1 and 2 as shown in FIG. 4. If there is some damage and the replacement of partial of the valve is needed. For example, only one leaflet is needed to be replaced. Therefore, the damaged leaflet is first removed per procedure. Then, portion of the pre-inserted valve will be attached to the right location, the damaged leaflet will be retracted

Use an index matching IR scope to see the structure in blood stream and suture or repair the valve leaflet one by one. The leaflet can be pulled to the site as described in section 2 using the material transfer mechanism indicated in FIG. 1e to transfer the valve leaflet.

Percutanueous method for repairing or replacing a pulmonary valve in a beating heart surgery is similar to arotic valve operation except the system is introduced through fermory vein then the interior vena cava.

3. Mitral Valve Replacement and Repair-Minimally Invasive

During a minimally invasive repairing or replacing a mitral valve in a beating heart surgery, inserting the surface vacuum device similar to FIG. 3 into the chest cavity under a traspeturesous and a fiber optical scope or a thorascope using separate ports on the chest; attaching the device onto the left atrium through a vacuum; insert a knife through the slit on the top of the device. After making an incision, retract the knife. Insert the mitrial valve separation unit and attach the elements to responding area.

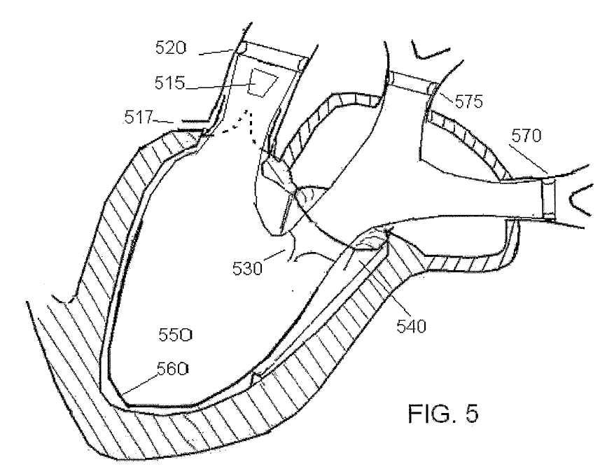

Depicted in FIG. 5 is an embodiment of the invention showing one possible configuration of a mitral separation element after deployment. It has a temporary aortic valve 510 (if the aortic valve 510 is not function well) and an associated balloon 520 to hold one end of the device in the aorta. There are holes 515 below the aorta balloon to allow blood to flow into coronary arteries 517. There is another temporary valve 530 replace the mitral valve 540 function, but below the mitral valve closing line. This allows the inspection of the mitral valve before or after repair. There are two attachment balloons just outside the left atrium to hold the other ends of the device thus to separate the mitral valve and blood stream to allow beating heart surgery while have best view.

Another feature of this device is that on the wall of the ventricle chamber 550, there is at least one elastic element 560 to extent the chamber during the expanding of the left ventricle to pump the blood from the atrium to the left ventricle.

Similarly, a minimally invasive method for repairing or replacing a tricuspid valve in a beating heart surgery is similar to the mitral repairing and replacement except the catheter is inserted from the right atrium

A percutanueouly method for repairing or replacing mitral valve in a beating heart surgery can be done by inserting device through the femoral artery. A percutanueouly method for repairing or replacing tricuspid valve in a beating heart can be done through the femoral vein.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A device for use in beating heart cardiac surgery, said device comprising:
(a) a valve separation unit;
(b) a visualization system;
(c) a surgical system; and
(d) a prosthetic valve or valve reinforcement ring element.

2. The device according to claim 1, wherein said valve separation unit is selected from a group comprising an aortic valve separation unit, a mitral valve separation unit, a tricuspid valve separation unit, and a pulmonary valve separation unit.

3. The device according to claim 2, wherein said aortic valve separation unit comprises:
(a) a left ventricular side attachment element;
(b) a temporary aortic valve;
(c) a connecting tube;
(d) a coronary perfusion element; and
(e) an ascending aorta attachment element.

4. The device according to claim 3, wherein the ventricular side attachment element and the ascending aorta attachment element are balloons.

5. The device according to claim 3, wherein the temporary aortic valve is at the center of the ventricular side aortic valve attachment element.

6. The device according to claim 3, wherein the coronary perfusion element is attached to the connecting tube that introduce the blood from the left ventricle to the coronary arteries.

7. The device according to claim 3, wherein the connecting tube connects the ventricular side aortic valve attachment element and the ascending aorta attachment element that provides a central pass way for the blood flow.

8. The device according to claim 3, wherein the aorta attachment element and the ascending aorta attachment element prevent blood flow into the separated valve area upon deployment.

9. The device according to claim 3, wherein the ventricular side attachment element and the ascending aorta attachment element are elastic membranes.

10. The device according to claim 2, wherein said pulmonary valve separation unit comprises:
(a) a right ventricular side attachment element;
(b) a temporary pulmonary valve;
(c) a connecting tube; and
(d) an ascending pulmonary artery attachment element.

11. The device according to claim 10, wherein the right ventricular side attachment element and ascending pulmonary artery attachment element are balloons.

12. The device according to claim 10, wherein the temporary valve is at the center of the right ventricular side attachment element.

13. The device according to claim 10, wherein the connecting tube connects the ventricular side attachment element and the ascending pulmonary artery attachment element that provides a central pass way for the blood flow.

14. The device according to claim 10, wherein the right ventricle attachment element and the pulmonary artery attachment element prevents blood flow into the separated valve area upon deployment.

15. The device according to claim 10, wherein the right ventricle attachment element and ascending pulmonary artery attachment element are elastic membranes.

16. The device according to claim 2, wherein said mitral valve separation element comprises:
(a) an aorta side attachment element;
(b) a temporary mitral valve;
(c) a ventricle chamber;
(d) a left atrium side attachment element; and
(e) an expansion element of the ventricle chamber

17. The device according to claim 16, wherein the aorta side attachment element secures the attachment to the inside wall of aorta.

18. The device according to claim 16, wherein the temporary mitral valve allows the blood flow from the left atrium to the left ventricle and then to the aorta.

19. The device according to claim 16, wherein the left ventricle chamber is fitted into the internal wall of the left ventricle upon activation.

20. The device according to claim 16, wherein the atrium side attachment element prevents blood flow from the pulmonary veins into separated mitral valve area.

21. The device according to claim 16, wherein the atrium side attachment element comprises a plurality of balloon or elastic membranes.

22. The device according to claim 2, wherein said tricuspid valve separation element comprises:
(a) a pulmonary side attachment element;
(b) a temporary tricuspid valve;
(c) a right ventricle chamber;
(d) a right atrium side attachment element; and
(e) an expansion unit.

23. The device according to claim 22, wherein the aorta side attachment element secures the attachment to the inside wall of pulmonary.

24. The device according to claim 22, wherein the temporary tricuspid valve allows the blood flow from the right atrium to right ventricle and then the pulmonary artery.

25. The device according to claim 22, wherein the right ventricle chamber is fitted into the internal wall of the right ventricle upon activation.

26. The device according to claim 22, wherein the atrium side attachment elements prevent blood flow from the superior vena cava and inferior vena cava vein into separated tricuspid valve area.

27. The device according to claim 22, wherein the right atrium side attachment element comprises a plurality of balloons or elastic vacuum activated membranes.

28. The device according to claim 1, wherein said visualization element is selected from a group comprising endoscope, fiberscope, and index matching IR fiber scope.

29. The device according to claim 1, wherein said surgical system comprises:
(a) a cutting element;
(b) a debris removal system having washing and aspiration element; (c) a suturing and attachment system;
(d) a local stabilizer;
(e) a heart open assistant device; and
(f) a material transfer mechanism

30. The device according to claim 29, wherein said cutting element further comprises a high speed cutting element and a manual cutter that can be inserted to the desirable locations minimal invasively.

31. The device according to claim 29, wherein said debris removal system further comprises first fluid introducing element and a fluid aspiration element.

32. The device according to claim 29, wherein said suturing and attachment system can be inserted to the desirable locations minimal invasively.

33. The device according to claim 29, wherein said suturing and attachment system is electrically or manually operated.

34. The device according to claim 29, wherein said heart opening system is a vacuum activated chamber can be inserted to the desirable locations minimal invasively through the chest and attached to heart surface to allow the incision to be made on the heart for insertion of the valve separation unit.

35. The device according to claim 29, wherein said local heart stabilizer is a holder that reduce the vibration of the surgical area through a negative pressure contacting point with the tissue and a rigid segment fixed outside the body.

36. The device according to claim 29, wherein said material transfer mechanism is a suture loop with one end at the surgical site that can be used to transfer materials and tools from outside the body to surgical site or vis versa

37. The device according to claim 1, wherein said valve reinforcement ring element is an annuloplasty ring.

38. The device according to claim 1, wherein said prosthetic valve is selected from a group comprising an aortic valve, a mitral valve, a tricuspid valve, and a pulmonary valve.

39. The device according to claim 1, wherein said prosthetic valve or valve reinforcement ring element can be pre-attached to the catheter devices or be transferred to the replacement site during surgery.

40. A method for repairing or replacing a heart valve minimal invasively in a beating heart surgery, said method comprising:
(a) inserting a devices of claim 34 into the chest under a traspeturesous and a fiber optical scope or a thorascope using separate ports on the chest;
(b) attaching the device of claim 34 onto the heart through a vacuum;
(c) inserting a knife to the slit on the top of the device;
(d) making an incision on the heart and retracting the knife;
(e) inserting a valve separation element of claim 3;
(f) activating the device for the surgery to establish alternative blood flow;
(g) washing and cleaning the area;
(h) repairing the heart valve removing the heart valve and suturing the prosthetic valve;
(i) extracting the device; closing the heart and all the wounds;

41. A method for repairing or replacing a heart valve-percutaneously in a beating heart surgery, said method comprising:
(a) inserting a device of claim 1 that comprises a valve separation unit into the heart through the femoral artery or vein and advanced percutaneously under a fiber optical scope;
(b) activating the device to establish alternative blood flow;
(c) repairing the heart valve or replacing the heart valve using prosthetic valve or ring;
(d) extracting the device; and
(e) closing all the wounds.

42. The method according claim 41 or claim 42, wherein the heart valve is selected from a group consisting of an aortic valve, a mitral valve, a tricuspid valve, and a pulmonary valve.

43. The The method according claim 41 or claim 42, wherein the heart attachment device can be place on arota, pulmonary artery, atrium, or ventricle for inserting the valve separation element.
